(19) United States
(12) Patent Application Publication
(10) Pub. No.: US 2018/0151253 A1
(43) Pub. Date: May 31, 2018

(54) ACCELERATED CLINICAL TRIAL DESIGN AND RECRUITMENT

(71) Applicant: PCRS Network, LLC, Cheyenne, WY (US)

(72) Inventor: John P. Neal, Las Vegas, NV (US)

(21) Appl. No.: 15/821,455

(22) Filed: Nov. 22, 2017

Related U.S. Application Data
(60) Provisional application No. 62/426,733, filed on Nov. 28, 2016.

(51) Int. Cl. G16H 10/20

(52) U.S. Cl. CPC G16H 10/20

(57) ABSTRACT

Accelerated clinical trial design and recruitment is disclosed. In an example, participants and clinical research sites are pre-identified and prequalified for clinical trials. Potential participant(s) and clinical research site(s) may be matched for a particular clinical trial. Patient records for the potential participants are selectively identified and de-identified to make a selection for the particular clinical trial. Individual identities of the identified participant(s) are made known only to the participant(s) and the clinical research site(s) that provided their record and/or their healthcare providers in compliance with applicable laws and regulations to facilitate effective clinical trial protocol and study design.

### PRIORITY CLAIM

This application claims the priority benefit of U.S. Provisional Patent Application No. 62/426,733 filed Nov. 28, 2016 of John P. Neal titled “Accelerated Clinical Trial Design and Recruitment Platform,” hereby incorporated by reference in its entirety as though fully set forth herein.

### BACKGROUND

Clinical trials are required before a treatment, therapy, or drug can be approved for use on general population patients. There are typically several phases to clinical trials. Early phases of clinical trials ensure the treatment is safe and may be conducted on animals (e.g., mice) in a controlled laboratory setting. Later phases help determine if the proposed new treatment, therapy, or drug is better than the current standard treatment, and to ensure it is safe for human patients. Each phase of a clinical trial has to be carefully designed to provide the requisite information about the new treatment, including but not limited to safety and efficacy (how well it works).

When creating a clinical trial design, it is important to determining “Inclusion” and “Exclusion” criteria for the people to participate in the clinical trial, identify potential volunteers or participants, and identify sites capable of conducting those studies (e.g., research hospitals and clinics). In the past, this has typically included everything from educated guesses to a variety of “home-grown” models based on past experience. Even commercially available analysis and tools are typically algorithms based on educated guesses and past experience.

Examples of identifying participants and sites for conducting clinical trials include attempting to identify geographic concentrations of patients suffering from the general disease state the clinical trial addresses, and then locating clinical research sites within those geographies that may be able to conduct the clinical trial. Other examples of identifying participants and sites for conducting clinical trials include sending feasibility questionnaires or surveys, and then conducting evaluations to try to determine which participants and/or clinical research sites are most probable to participate in the clinical trial.

As can be readily appreciated, the current techniques for designing clinical trials are time consuming, inefficient, rife with error due to broad speculation and uncertainty, and difficult to find participants and clinical sites that meet the criteria and/or are willing to participate.

### BRIEF DESCRIPTION OF THE DRAWINGS

FIG. 1 is a high-level illustration of an example environment which may implement accelerated clinical trial design and recruitment.

FIG. 2 is a process flow diagram showing an example of accelerated clinical trial design and recruitment.

FIGS. 3-5 are flowcharts of example operations for accelerated clinical trial design and recruitment.

### DETAILED DESCRIPTION

The accelerated clinical trial design and recruitment platform disclosed herein enables a new way of designing clinical trials and recruiting participants into clinical trials that reduces clinical trial delays by identifying individuals who meet the clinical trial inclusion and exclusion criteria who have already indicated a desire to participate in clinical trials, and who may be affiliated with a prequalified clinical research site. The accelerated clinical trial design and recruitment platform reduces delays in completing enrollment into clinical trials.

Accelerated clinical trial design and recruitment is disclosed. In an example, participants and clinical research sites are pre-identified and prequalified for clinical trials. Potential participant(s) and clinical research site(s) may be matched for a particular clinical trial. Patient records for the potential participants are selectively identified and de-identified (e.g., blinded or anonymized) to make a selection for the particular clinical trial. Individual identities of the identified participant(s) are made known only to the participant(s) and the clinical research site(s) that provided their record and/or their healthcare providers in compliance with applicable laws and regulations to facilitate effective clinical trial protocol and study design.

Because all of the people in the accelerated clinical trial design and recruitment database have pre-indicated an interest in participating in clinical trials, the conversion rate is extraordinarily high. In an example, potential participants have either self-identified and entered their data, in which case they are assigned to a prequalified site. In another example, participant information is provided by a prequalified clinical research site, so there is an existing relationship between the participant and the clinical research site.

In an example, the accelerated clinical trial design and recruitment platform actively manages volunteer engagement so as to keep the information on potential participants and clinical research sites fresh, and to keep the volunteer involved, making the relationship “sticky” (e.g., reduced possibility of a poor match).

Before continuing, it is noted that as used herein, the terms “includes” and “including” mean, but is not limited to, “includes” or “including” and “includes at least” or “including at least.” The term “based on” means “based on” and “based at least in part on.”

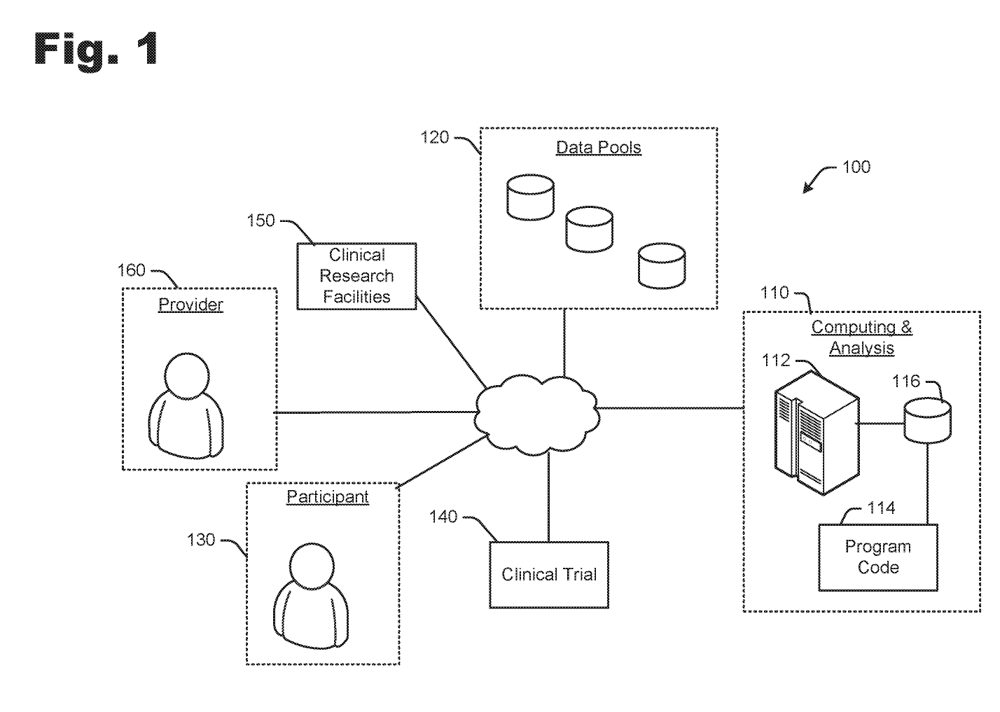

FIG. 1 is a high-level illustration of an example environment 100 which may implement accelerated clinical trial design and recruitment. System 100 may be implemented with any of a wide variety of devices, such as, but not limited to, data processors, servers, mobile devices, and cloud computing devices, to name only a few examples. Computing devices may include memory, storage, and a degree of data processing capability at least sufficient to manage a communications connection either directly with one another or indirectly (e.g., via a network). At least one of the computing devices is also configured with sufficient processing capability to execute program code.

In an example, the system 100 includes computing and analysis platform 110. For purposes of illustration, the platform 110 may include processing services executing on a server computer 112. The processing services may execute machine readable instructions or program code 114 stored in computer-readable storage 116. Example processing services provided by the platform 110 may include, interfaces to processors, application engines and associated interfaces, and related hardware and software support infrastructure.

In an example, the platform 110 has access to data pools 120. Data pools may be stored locally and/or remotely (e.g., in the cloud) and accessed/provided over a communications network. Data pools 120 include data that undergoes at least some degree of processing to transform the data according to the techniques described herein.

The data pools 120 may include any type and amount of content. In an example, the data pools 120 include sensitive patient information, and is handled according to applicable laws and regulations to protect patient privacy. In an example, patient information may be protected by disassociating patient identification from clinical information (de-identification). In another example, patient information may be provided only to the patient, the patient provider (e.g., a doctor), and/or other person designated by the patient. There is no limit to the type or amount of content that may be processed. In addition, the content may include unprocessed or unstructured (e.g., “raw”) data, or the content may undergo at least some level of processing.

As mentioned above, the techniques described herein may be embodied at least in part by program code 114 executed by any suitable computing device. Program code used to implement accelerated clinical trial design and recruitment can be better understood with reference to the following discussion of various example operations. However, the operations described herein are not limited to any specific implementation with any particular type of device structure. In an example, the program code may be implemented in machine-readable instructions. The machine-readable instructions may be stored on a non-transient computer readable medium and are executable by one or more processor to perform the operations described herein. The operations may be executed as self-contained modules. These modules can be integrated within a self-standing tool, or may be implemented as agents that run on top of an existing device structure. It is noted, however, that the computing environment described herein is provided only for purposes of illustration of an example operating environment, and is not intended to limit implementation to any particular system.

In an example, the accelerated clinical trial design and recruitment platform may be implemented as a Software as a Service (SaaS) system utilizing information technology in an innovative combination of applications that engage potential participants 130 (e.g., via a web browser or a mobile App) to accelerate completion of clinical trials 140. However, the operations described herein are not limited to any particular device implementation.

In an example, the accelerated clinical trial design and recruitment platform starts the volunteer recruitment process where current methodologies fail, by identifying specific volunteers 130 for participation in clinical trials 140. The accelerated clinical trial design and recruitment platform 100 facilitates better study protocol design and selection of research sites by using the Personal Health Information (PHI) of people who have already indicated an interest in participating in research, creating real-world, executable clinical trials, and selecting sites or clinical research facilities or sites 150 based on access to pre-identified volunteers 130 fitting the study requirements.

The SaaS platform may be implemented for designing clinical research trials, and then recruiting volunteers 130 into the trials faster. Pharmaceutical companies, CRO's, Clinical Research Sites, and Volunteers are engaged via a web browser or a smartphone App. The methodology at the core of the platform starts the volunteer recruitment process at the point of protocol design, rather than after sites 150 are selected and initiated to begin recruiting. The database 120 provides access to, and aggregation of, the study volunteer databases 120 of multiple clinical research sites 150

The accelerated clinical trial design and recruitment platform 100 quickly and accurately identifies volunteers for studies, matches them to prequalified research sites 150, and facilitates all aspects of enrollment in studies 140. Volunteer data is aggregated across multiple pre-vetted research sites and adds to the database 120 for volunteers opting in.

The accelerated clinical trial design and recruitment platform 100 enables rapid clinical trial design and subsequent recruitment of study volunteers based on accessing personal health information of people who have pre-volunteered or opted-in or have not opted out to participate in clinical trials, with communication of confirmation of interest and qualification facilitated within the platform. Volunteers are assigned to prequalified clinical research sites 150, that can quickly confirm qualification and interest of the volunteer, schedule screening events, and communicate with volunteers and their healthcare providers 160 through the platform 100.

In an example, a Sponsor determines they need at least 500 subjects to complete a study, with a target of 10 subjects enrolled at each Site. Using the Sponsor's planned I/E, the accelerated clinical trial design and recruitment platform identifies individuals from the database (which may also access each patient's Electronic Health Record (EHR)) who have already opted in to participate in clinical trials, who appear to meet all I/E, and who are already assigned to a Site.

Via the accelerated clinical trial design and recruitment platform, volunteers are contacted to verify they are still interested in participating in clinical research, they are not currently participating in any other trials, and to update their medical history. Based on volunteer concentrations, and conversion metrics by Site, the accelerated clinical trial design and recruitment platform determines the actual number of Sites needed (based on pilot cases to date, expected to be less than half the Sponsors original estimate.)

It is noted that the examples shown and described herein are provided to illustrate example implementations. Still other examples may also be implemented.

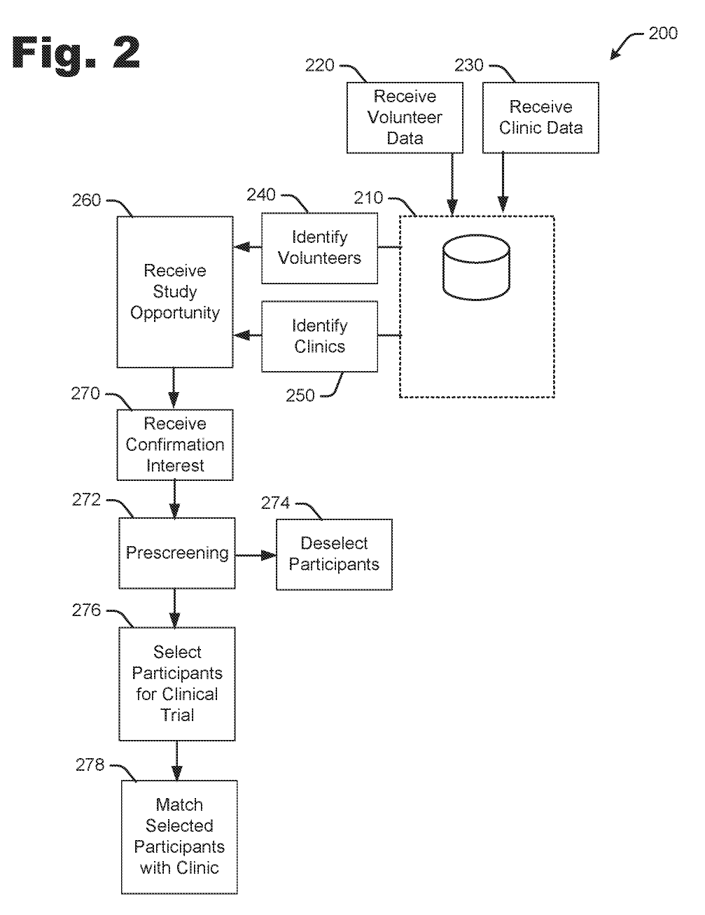

FIG. 2 is a process flow diagram 200 showing an example of accelerated clinical trial design and recruitment. The accelerated clinical trial design and recruitment platform may include a database 210 aggregating clinical trial volunteer data 220 and clinical research sites 230, e.g., from online submission of personal health information, including access via electronic means such as a website, electronic forms, smart device applications, and other means. The platform provides query functions that facilitate identifying volunteers 240 and clinics 250, e.g., based on but not limited to geographic concentrations, travel distances, and times to assigned clinical research sites.

The database(s) 210 may be accessed as needed to design clinical research protocols, identify potential participants 240 (volunteers) in studies, identify potential clinical research sites 250 to conduct the studies, in addition to communicate with the volunteers, schedule screening events, establish interest in, and eligibility for, participating in clinical trials.

The accelerated clinical trial design and recruitment platform may include a suite of components comprising a database of volunteer personal health information obtained from clinical research sites and data provided directly or indirectly by people or guardians thereof interested in volunteering to participate in clinical research studies/trials.

Data collection may comprise one or more entry devices, smart devices, or electronic applications, wearable devices capable of recording and transmitting personal health information and medical events, such as, but not limited to, blood pressure, heart rate, blood glucose, balance, exercise, hearing acuity, visual acuity, picture, video, weight, and body mass.

When a study opportunity is received 260, the data may be processed for individual identification of prospective volunteers for clinical research studies/trials utilized to design clinical trials, identify locations of prospective volunteers, select clinical research sites to conduct the clinical trials, communicate between the volunteers, clinical research sites, and the database for purposes of maintaining up to date contact, personal health information, opt-in/opt-out status, prior and current clinical trial participation, and other relevant data.

In an example, volunteers are contacted to confirm interest 270. Those volunteers that are interested are prescreened 272 based on various criteria. Participants may be deselected 274. Participants who are selected 276 may be matched with a clinic for the study. It is noted that some or all of this process may be iterative.

In an example, an accelerated clinical trial design and recruitment platform includes a database of pre-identified people whose records are aggregated from prequalified clinical research sites or are self-provided, who have indicated they would like to participate in a clinical trial if one meets their needs. Program code may pre-assign those people to clinical research sites that have been prequalified to be capable of conducting clinical trials. The program code may confirm volunteer interest in participating in a clinical trial of a particular design via the integrated platform of the invention. The program code may provide two-way communication between the volunteer and the clinical research site with which they are affiliated to facilitate communications related to participating in clinical research. The program code enables database records to be selectively de-identified to make individuals identities known only to themselves and the clinical research site that provided their record and/or their healthcare providers. The program code may provide communication between healthcare product providers and volunteers in the database on a de-identified basis. The program code enables database analysis on an identified and/or de-identified basis in compliance with applicable laws and regulations to facilitate effective clinical trial protocol and study design.

Each patient/volunteer may receive a unique ID. The Site ID may be prepended to the Site provided patient/volunteer ID. Patient/volunteer identifying information, including contact information, may be de-identified so as to protect Site's business information and the patient's identity from discovery by users of the platform. De-identified records remain the property of providing Site, which provides owner of the platform an exclusive license to use the data for purposes of providing services anticipated by the platform

Patient engagement may be enhanced by frequent contact with individuals within the platform database to create a relationship with the individual and make that relationship “sticky”, such that the individual feels an affinity for the platform and the Site with which they are affiliated/assigned. In an example, the platform facilitates frequent contact with individuals in the database via one of many “push” technologies, providing information beneficial to the individuals regarding treatments, medications, devices, etc., and information about activities, events, articles, future clinical trials, currently enrolling clinical trials, and/or other information relevant to the health and disease states of the individuals. Also in an example, the platform facilitates communication between each individual and the Site from which the individual's records originated or to which the individual has been assigned via one or more means including, but not limited to, push messages via an app, text message, email, automated phone contact, live chat, or other methodologies. Individuals may also request information and or contact via the platform to initiate “conversation” with a representative of the platform or the Site with which they are affiliated. Such conversations may occur as chats, texts, emails, phone calls, or other means.

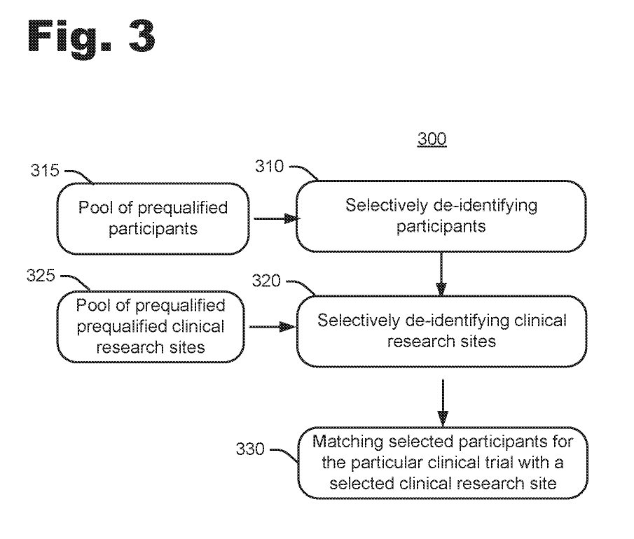

FIGS. 3-5 are flowcharts of example operations for accelerated clinical trial design and recruitment. Before continuing, it should be noted that the examples described above are provided for purposes of illustration, and are not intended to be limiting. Other devices and/or device configurations may be utilized to carry out the operations described herein.

FIG. 3 illustrates an accelerated clinical trial design and recruitment method 300. Operation 310 includes selectively de-identifying participants from a pool 315 of prequalified participants for a particular clinical trial. Operation 320 includes selectively de-identifying clinical research sites from a pool 325 of prequalified clinical research sites for a particular clinical trial.

Operation 330 includes matching selected participants for the particular clinical trial with a selected clinical research site. In an example, individual identities of the identified participants are made known only to the selected participants and the selected clinical research site in compliance with applicable laws and regulations to facilitate effective clinical trial protocol and study design.

In an example, operations may also include validating participant interest.

In an example, operations may also include prequalifying a clinical research site. In an example, operations may also include prequalifying a participant.

In an example, operations may also include maintaining the pool of prequalified clinical research sites. In an example, operations may also include maintaining the pool of prequalified participants.

In an example, operations may also include providing a unique identification to each prequalified participant.

In an example, operations may also include receiving a request for the particular clinical trial.

In an example, operations may also include prescreening volunteers for the pool of prequalified participants. In an example, operations may also include prescreening volunteers for the pool of prequalified clinical research sites.

In an example, operations may also include scheduling a prequalified participant with a prequalified clinical research site for the particular clinical trial.

In an example, operations may also include determining effective study parameters for the particular clinical trial.

In an example, operations may also include targeting potential participants based on the effective study parameters for the particular clinical trial.

In an example, operations may also include generating what-if scenarios based on iterations of inclusion and exclusion Boolean searches to identify potential participants based on the effective study parameters for the particular clinical trial.

In an example, operations may also include matching potential participants with potential clinical research sites for the particular clinical trial based on at least proximity.

In an example, operations may also include matching potential participants with potential clinical research sites for the particular clinical trial based on at least health condition of the potential participants and specialty of the potential clinical research site.

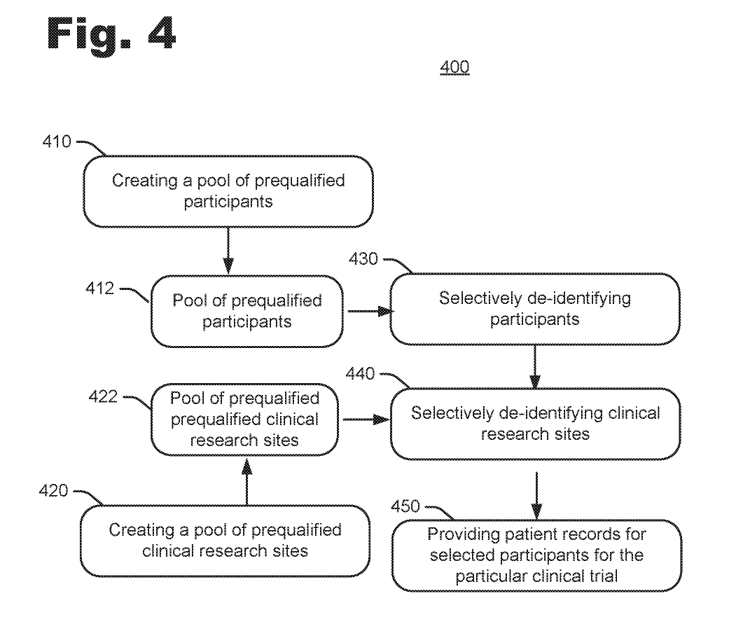

FIG. 4 illustrates an accelerated clinical trial design and recruitment method 400. Operation 410 includes creating a pool 412 of prequalified participants for a particular clinical trial. Operation 420 includes creating a pool 422 of prequalified participants and a pool 414 prequalified clinical research sites for a particular clinical trial.

Operation 430 includes selectively de-identifying participants from the pool of potential participants. Operation 440 includes selectively de-identifying clinical research sites from the pool of potential clinical research sites.

Operation 450 includes providing patient records for selected participants for the particular clinical trial. In an example, individual identities of the identified participants are made known only to the identified participants and the clinical research sites in compliance with applicable laws and regulations to facilitate effective clinical trial protocol and study design.

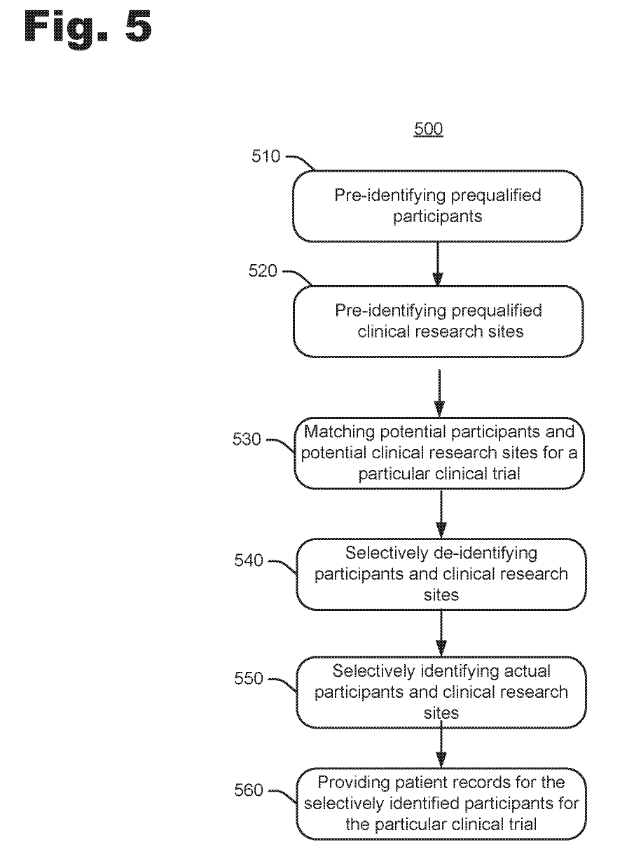

FIG. 5 illustrates an accelerated clinical trial design and recruitment method 500. Operation 510 includes pre-identifying prequalified participants for clinical trials. Operation 520 includes pre-identifying prequalified clinical research sites for clinical trials. Operation 530 includes matching potential participants and potential clinical research sites for a particular clinical trial. Operation 540 selectively de-identifying participants and clinical research sites from the matched potential participants and potential clinical research sites. Operation 550 includes selectively identifying actual participants and clinical research sites from the matched potential participants and potential clinical research sites. Operation 560 includes providing patient records for the selectively identified participants for the particular clinical trial. In an example, individual identities of the identified participants are made known only to the identified participants and the clinical research sites in compliance with applicable laws and regulations to facilitate effective clinical trial protocol and study design.

It is noted that the examples shown and described are provided for purposes of illustration and are not intended to be limiting. Still other examples are also contemplated.

1. An accelerated clinical trial design and recruitment method, comprising:
selectively de-identifying participants and clinical research sites from a pool of prequalified participants and a pool of prequalified clinical research sites for a particular clinical trial; and
matching selected participants for the particular clinical trial with a selected clinical research site, wherein individual identities of the identified participants are made known only to the selected participants and the selected clinical research site in compliance with applicable laws and regulations to facilitate effective clinical trial protocol and study design.

2. The accelerated clinical trial design and recruitment method of claim 1, further comprising validating participant interest.

3. The accelerated clinical trial design and recruitment method of claim 1, further comprising prequalifying a clinical research site.

4. The accelerated clinical trial design and recruitment method of claim 1, further comprising prequalifying a participant.

5. The accelerated clinical trial design and recruitment method of claim 1, further comprising maintaining the pool of prequalified clinical research sites.

6. The accelerated clinical trial design and recruitment method of claim 1, further comprising maintaining the pool of prequalified participants.

7. The accelerated clinical trial design and recruitment method of claim 1, providing a unique identification to each prequalified participant.

8. The accelerated clinical trial design and recruitment method of claim 1, further comprising receiving a request for the particular clinical trial.

9. The accelerated clinical trial design and recruitment method of claim 1, further comprising prescreening volunteers for the pool of prequalified participants.

10. The accelerated clinical trial design and recruitment method of claim 1, further comprising prescreening volunteers for the pool of prequalified clinical research sites.

11. The accelerated clinical trial design and recruitment method of claim 1, further comprising scheduling a prequalified participant with a prequalified clinical research site for the particular clinical trial.

12. The accelerated clinical trial design and recruitment method of claim 1, further comprising determining effective study parameters for the particular clinical trial.

13. The accelerated clinical trial design and recruitment method of claim 12, further comprising targeting potential participants based on the effective study parameters for the particular clinical trial.

14. The accelerated clinical trial design and recruitment method of claim 12, further comprising generating what-if scenarios based on iterations of inclusion and exclusion Boolean searches to identify potential participants based on the effective study parameters for the particular clinical trial.

15. The accelerated clinical trial design and recruitment method of claim 1, further comprising matching potential participants with potential clinical research sites for the particular clinical trial based on at least proximity.

16. The accelerated clinical trial design and recruitment method of claim 1, further comprising matching potential participants with potential clinical research sites for the particular clinical trial based on at least health condition of the potential participants and specialty of the potential clinical research site.

17. An accelerated clinical trial design and recruitment method, comprising:
creating a pool of prequalified participants and prequalified clinical research sites for a particular clinical trial;
selectively de-identifying participants and clinical research sites from the pool of potential participants and potential clinical research sites;
providing patient records for selected participants for the particular clinical trial, wherein individual identities of the identified participants are made known only to the identified participants and the clinical research sites in compliance with applicable laws and regulations to facilitate effective clinical trial protocol and study design.

18. The accelerated clinical trial design and recruitment method of claim 17, further comprising generating what-if scenarios based on iterations of inclusion and exclusion Boolean searches to identify potential participants based on the effective study parameters for the particular clinical trial.

19. An accelerated clinical trial design and recruitment method, comprising:
pre-identifying prequalified participants and clinical research sites for clinical trials;
matching potential participants and potential clinical research sites for a particular clinical trial;
selectively de-identifying participants and clinical research sites from the matched potential participants and potential clinical research sites;
selectively identifying actual participants and clinical research sites from the matched potential participants and potential clinical research sites; and
providing patient records for the selectively identified participants for the particular clinical trial, wherein individual identities of the identified participants are made known only to the identified participants and the clinical research sites in compliance with applicable laws and regulations to facilitate effective clinical trial protocol and study design.

20. The accelerated clinical trial design and recruitment method of claim 19, further comprising generating what-if scenarios based on iterations of inclusion and exclusion Boolean searches to identify potential participants based on the effective study parameters for the particular clinical trial.
